(19) United States
(12) Patent Application Publication
(10) Pub. No.: US 2004/0126386 A1
(43) Pub. Date: Jul. 1, 2004

(54) P53 vaccine

(76) Inventor: Arnold J. Levine, Princeton, NJ (US)

Correspondence Address: Laura S. Weiss, ImClone Systems Incorporated, 180 Varick Street, New York, NY, 10014, US

(21) Appl. No.: 10/734,476

(22) Filed: Dec. 12, 2003

Related U.S. Application Data
(63) Continuation of application No. 09/946,549, filed on Sep. 6, 2001, now abandoned.
Application No. 09/946,549 is a continuation of application No. 08/366,512, filed on Dec. 30, 1994, now abandoned.
Application No. 08/366,512 is a continuation-in-part of application No. 08/185,738, filed on Jan. 24, 1994, now abandoned.
Application No. 08/185,738 is a continuation-in-part of application No. 08/015,493, filed on Feb. 9, 1993, now abandoned.
Application No. 08/015,493 is a continuation-in-part of application No. 07/918,292, filed on Jul. 22, 1992, now abandoned.

(51) Int. Cl. A61K 39/00; C12N 5/08

(52) U.S. Cl. 424/185.1; 424/277.1; 435/372

(57) ABSTRACT

The subject invention provides a vaccine composition comprising a mutant or wild-type p53 protein in a form that, when presented to the immune system of a mammal, induces an effective immune response, i.e., either on the surface of an antigen presenting cell or combined with a pharmaceutically acceptable adjuvant. Further, the subject invention provides a method of inhibiting the growth of tumors in mammals comprising treating a mammal with an immunologically effective amount of a vaccine comprising the mutant or wild-type p53 protein.

This application is a continuation-in-part of Ser. No. 08/185,738, filed Jan. 24, 1994, which is a continuation-in-part of Ser. No. 08/015,493, filed Feb. 9, 1993, which in turn is a continuation-in-part of Ser. No. 07/918,292, filed Jul. 22, 1992, both of which are incorporated herein by reference.

The present invention is directed to a vaccine for treating cancer. The vaccine comprises a p53 protein as the immunogen.

The p53 gene, which is found on chromosome 17p of the human genome, is a tumor suppressor gene in its wild-type state. A review article by Levine et al. entitled “The p53 Tumor Suppressor Gene” appears in Nature 351, 453-456 (1991).

More than 50% of human tumors contain cells expressing a mutant form of the p53 gene. In many tumors, one allele of the p53 gene contains a point mutation that encodes a mutant form of the protein while the other allele is partially or totally lost. This pattern is observed, for example, in approximately 70-80% of colon cancers, 50% of breast cancers, and 50% of lung cancers including 100% of small cell lung cancers. Suggestions have been made to diagnose cancers by detecting the loss of wild type p53 (see Vogelstein et al., European Patent Application 390,323 and Baker et al., Science 244, 217-221 (1989).

The position or location of the point mutation in the p53 gene differs in different types of tumors. For example, 50% of the hepatocellular carcinomas in humans exposed to hepatitis B and aflotoxin contain p53 mutations at codon 249; lung tumors appear to contain mutations preferentially at codons 154 and 273; colon tumors have multiple independent mutations at codons 175, 248, and 273. Evidence has been presented that various phenotypes, including the severity and nature of cancer and pre-cancer states, can be distinguished by determining the site of p53 mutations. See Levine et al., International Application No. PCT/US91/04608, filed Jun. 27, 1991.

Approximately 10-20% of humans with cancers have tumors that produce antibodies directed against the p53 protein; de Fromentel et al., International Journal of Cancer 39, 185-189 (1987); Crawford et al., International Journal of Cancer 30, 403-408 (1982). The presence of these antibodies suggests that class II receptors of the human HLA or the murine H-2 locus can present peptide antigens of p53 to the CD-4 helper T-cell and B-cell system, resulting in an immune response. Antibodies are not, however, believed to be effective ant-tumor agents. Therefore, the presence of anti-p53 antibodies in humans with cancer does not suggest the possibility of cancer patients producing an effective anti-tumor immune response.

There are reports that animals immunized with a tumor antigen are protected against the same antigen. Thus, immunizing animals with simian virus 40 (SV40) large T antigen can protect against subsequent challenges with live tumorigenic SV40-transformed cells; see Tevethia et al., Cold Spring Harbor Symp. Quant Biol. 44, 235-242 (1980).

Similarly, Frey and Levine have reported that rats immunized with an irradiated p53-plus-ras-transformed Fisher rat cell line, designated B3, were protected from subsequent tumor challenge with the same live cell. The p53-plus-ras-transformed rat cell lines were reported to express a tumor-specific transplantation rejection antigen that is common to 85% of independently derived p53-plus-ras-transformed cell lines. Frey and Levine presented evidence that the p53 protein is not the tumor-specific transplantation rejection antigen, and does not protect against challenge by B3 cells; see Journal of Virology 63, 5440-5444 (1989).

Current cancer treatments involve cytotoxic agents, such as chemical compounds and radiation, that are insufficiently specific to tumor cells. There is a need for more specific treatments that do not affect normal cells. There is a particular need for cancer treatments that result from stimulating a patent's own immune system.

### SUMMARY OF THE INVENTION

These and other objects as will be apparent to those having ordinary skill in the art have been met by providing a vaccine composition comprising a mutant or wild-type p53 protein in a form that, when presented to the immune system of a mammal, induces an effective immune response.

The invention further provides a method of inhibiting the growth of tumors in mammals comprising treating a mammal with an immunologically effective amount of a mutant or wild-type p53 protein.

### DESCRIPTION OF THE FIGURES

FIG. 1: Depicted is the tumor diameter in mm of (10)3-175.1 and (10)3-273.1 induced tumors over time in days. (10)3-175.1 and (10)3-273.1 cells were injected s.c. at 1*10⁷ cells per mouse into 10 mice each. Numbers correspond to individual mice.

FIG. 2: Immunoprecipitation of metabolically labeled SV-80 protein extract using 3 μl of mouse serum from untreated mice (mock; lane 2), mice injected with (10)3 cells (lanes 3, 4), mice injected with (10)3-175.1 cells (lanes 5, 6, 7, 8), mice injected with (10)3-273.1 cells (lanes 8, 9, 10) or p53-specific monoclonal antibody pab421 (lane 1). p53 and bound SV40 T antigen are coimmunoprecipitated only with serum from (10)3-273.1 injected mice (lanes 8, 9, 10). An autoradiograph of the SDS-PAGE of the immunoprecipitates is shown.

FIG. 3: Kaplan-Meier-Plot of percent tumor-free animals over time at various concentrations (10⁴, 10⁵, 10⁶, 5*10⁶, 10⁷) of (10)3-273.1.NT24 cells. The cells were injected s.c. into 3 BALB c/J mice each. (10)3-273.1.NT24 form progressively growing tumors in untreated mice which become necrotic within 10 days of initial appearance.

FIG. 4: Depicted is tumor diameter in mm of (10)3-273.1NT24 induced tumors over time/days. 1*10⁵ (10)3-273.1NT24 cells were injected s.c. into 10 mice each which were either non-immunized (mock) or immunized with 2 successive injections of 1*10⁶ mitomycin C treated (10)3 cells, (10)3-273.1 cells or (10)3-273.1NT24 cells.

FIG. 5: A) Kaplan-Meier-Plot of percent tumor-free animals over time at various concentrations (10⁴, 10⁵, 10⁶) of (10)3-tx4BT87 cells which were injected s.c. into 3 BALB c/J mice each. (10)3-tx4BT87 form progressively growing tumors in untreated mice which become necrotic within 10 days of initial appearance. B) Kaplan-Meier-Plot of percent tumor-free animals over time either non-immunized (n=10) or immunized with (10)3-273.1 cells (n=10) and challenged with at 10⁵ (10)3-tx4BT87 cells.

FIG. 6: Immunoprecipitation of in vitro translated human p53 using 3 μl of pooled serum drawn from mice shown in FIG. 4 at the time of injection. The autoradiograph of the SDS-PAGE of the immunoprecipitates is shown. Lane 1: immunoprecipitation of p53 and p53 breakdown products using monoclonal antibody pab421 lane 2, 3, 4, 5: immunoprecipitation with serum from nonimmunized mice, (10)3-273.1NT24, (10)3, (10)3-273.1 immunized mice respectively; lane 6 [¹⁴C]-labeled molecular weight markers (BRL).

FIG. 7: FACS-analysis of MHC-I H2-K^{d} or H2-K^{b} surface expression on (10)3, (10)3-273.1, (10)3-273.1N124 cells. P815 and P815:273 cells are used as controls. They are mouse mastocytoma cell lines and express high levels of H2-K^{d}. P815:273 express the human p53 allele mutated at amino acid 273.

FIG. 8: [⁵¹Cr]-release assay on lymphocytes isolated from mice which were injected with 10⁶ (10)3-273.1 cells (b,c,e-j) or normal mice (a,d). Either (10)3 cells (open circles) or (10)3-273.1 cells (closed circles) were used as target cells. Displayed is percent lysis at a given effector to target ratio (E:T). Each point represents the average 3 to 4 measurements. Spontaneous lysis was less than 20% of maximal lysis.

FIG. 9: Kaplan-Meier-Plot of percent tumor-free transgenic mice ( . . . ; total n=19) or non-transgenic littermates (-; total n=22) over time. Shown are 3 separate experiments in which matched siblings were injected s.c. with 106 (10)3-273.1 cells per mouse. The combined stratified Wilcox-Sum-Rank-test is p=0.001.

FIG. 10: Immunoblot analysis of p53 protein expressed in BCG bacteria. Bacteria were lysed in cytoplasmic lysis buffer. One mg of protein extract was analyzed in each experimental lane of this SDS-PAGE. The protein was transferred to nitrocellulose, incubated with antibody pab421 and peroxidase conjugated goat anti-mouse antibody (1:5000, Cappel), and developed with ECL (Amersham).

One-hundred ng human p53 protein purified from baculovirus extract was used as a positive control. Two out of two clones of wild-type and mutant p53 exon 5-11 express the respective 28 kD fragment. The upper bands might represent aggregates and are present at 1/10 the level of the specific expression product. One clone (lane 2) of the full-length p53 expression constructs expresses p53.

FIG. 11: Immunoprecipitation and subsequent Western analysis of recombinant human full-length p53 expressed in BCG bacteria. Bacteria were lysed in lysis buffer and 1 mg extract was immunoprecipitated using either pab421, pab1801 or a control antibody pab419. The immunoprecipitable material was subjected to SDS-PAGE analysis, transferred to nitrocellulose and detected with polyclonal rabbit anti-p53 antiserum (1:500 dilubon) and visualized with peroxidase conjugated anti-rabbit-IgG (Cappel, 1:5000 dilution) and ECL (Amersham). BCG-SN₃ expressed p53 under control of the BCG hsp60 promoter. p53 was immunoprecipitable by pabl801 directed against an N-terminal epitope and pab421 directed against a C-terminal epitope. Heat-shock of the bacteria did not increase the expression level. Untransformed BCG bacteria did not express p53. The secondary anti-rabbit-IgG had cross-reactivity to the IgG heavy and light chains of the monoclonal antibodies. More p53 was immunoprecipitated with pab421, which is the p53 antibody with the highest binding constant to p53 and which recognizes native and also denatured protein.

FIG. 12: Kaplan-Meyer plot of percent tumor-fee animals over time in days. Animals which did not develop progressive tumors were considered tumor-free. Graph A compares animals immunized with BCG expressing truncated (containing exons 5-11) wild-type (n=5) or mutant p53 (n=10) to mock, i.e. untreated, animals (n=5) challenged at the same time. Graph B compares animals immunized with BCG bacteria alone (n=10) to untreated animals (n=10).

FIG. 13: Graph of changes in tumor size, i.e. diameter over time in days. Graph A compares untreated animals (n=5) to mice immunized with BCG expressing truncated (containing exons 5-11) wild-type p53 (n=5). None of the latter developed tumors. Graph B shows animals immunized with BCG expressing truncated (containing exons 5-11) mutant p53. Two animals developed progressively growing tumors. Graph C shows animals immunized with BCG expressing full-length wild-type p53. All animals developed lesions, but the lesions did not grow.

FIG. 14: Immunization with ALVAC virus. Shown on the vertical axis is the time in days until the mice immunized with the respective vaccine (on the horizontal axis) developed tumors. Open circles represent individual mice, open squares the mean tumor-free survival time within each group±standard deviation. The lower p-values show the significance as calculated using a Mann-Whitney U Test between each subgroup and the vector alone. The upper p-values show the significance in tumor-free survival time compared to immunization with ALVAC vector alone for the groups of mice immunized with ALVAC expressing either human or murine p53 as calculated using a Mann-Whitney U Test.

### DETAILED DESCRIPTION OF THE INVENTION

The subject invention provides a vaccine composition comprising a mutant or wild-type p53 protein in a form that, when presented to the immune system of a mammal, induces an effective immune response. For example, the mutant or wild-type p53 protein may be present on the surface of an antigen presenting cell or liposome or combined with a pharmaceutically acceptable adjuvant.

For the purposes of the present specification, the term “wild-type p53 protein” means the nucleotide or amino acid sequence reported by Matashewski et al, EMBO J. 13, 3257-3262(1984); Zakut-Houri et al, EMBO J. 4, 1251-1255(1985); and Lamb and Crawford, Mol. Cell. Biol. 5, 1379-1385 (1986). The sequences are available from GenBank. Wild-type p53 includes a proline/arginine polymorphism at amino acid 72 and the corresponding nucleotide polymorphism.

The p53 protein may be mutated. The data shown in Example 1 demonstrate that overexpression of mutant p53 in experimental tumors can induce an immune response which is dependent on mutant p53 protein expression. This immune response constrained tumor growth of moderately tumorigenic cells ((10)3-273.1) and upon immunization resulted in tumor rejection of highly tumorigenic variants (10)3-273.1 NT24). Tolerance to p53 evoked by expression of a human p53 transgene impaired tumor immunity.

The p53 mutation is preferably at a position that is frequently found to be mutated in tumor cells, and that leads to inactivation of the wild-type p53 gene. The mutations may be either a single substitution or multiple (i.e. 2-20, preferably 2-10, more preferably 2-5) substitutions.

Suitable mutant human p53 genes are described in Levine, A. J. et al., The p53 Tumor Suppressor Gene, Nature 351:453-456 (1991). Most of the point mutations that occur in the p53 gene are missense mutations, giving rise to an altered p53 protein. The majority of mutations are clustered between amino-acid residues 130 and 290, and mostly localized in four “hot spot” regions of the protein, which coincide with the four most highly conserved regions of the p53 gene; see Nigro et al, Nature 342, 705-708 (1989). The four “hot spot” mutation regions are at codons 132-143; 174-179; 236-248; and 272-281. The frequency and distribution of these hot spots differ among cancers from different tissue types.

The wild-type p53 gene and protein are known, and may be obtained in natural or recombinant form by known methods. Such methods include isolating the protein directly from cells; isolating or synthesizing DNA encoding the protein and using the DNA to produce recombinant protein; and synthesizing the protein chemically from individual amino acids. Methods for obtaining the wild-type p53 gene and protein are described in Matlashewski et al, EMBO J. 13, 3257-3262 (1984); Zakut-Houri et al, EMBO J. 4, 1251-1255 (1985); and Lamb and Crawford, Mol. Cell. Biol. 5, 1379-1385 (1986). Mutants may be prepared from the wild-type p53 gene by site-directed mutagenesis; see, for example, Zoller and Smith, Nucl. Acids Res. 10, 6487-6500 (1982); Methods in Enzymology 100, 468-500 (1983); and DNA 3; 479-488 (1984).

The entire p53 gene or fragments of the p53 gene may, for example, be isolated by using the known DNA sequence to construct oligonucleotide probes. To do so, DNA restriction fragments are identified by Southern hybridization using labelled oligonucleotide probes derived from the known sequence.

Alternatively, p53-encoding DNA may be synthesized from individual nucleotides. Known methods for synthesizing DNA include preparing overlapping double-stranded oligonucleotides, filling in the gaps, and ligating the ends together.

The DNA prepared as described above may be amplified by polymerase chain reaction (PCR). Alternatively, the DNA may be amplified by insertion into a cloning vector, which is transfected into a suitable host cell, from which the p53 DNA may be recovered. See, generally, Sambrook et al, “Molecular Cloning,” Second Edition, Cold Spring Harbor Laboratory Press (1987).

Recombinant methods well known in the art may be used for preparing the protein. Briefly, p53-encoding DNA is inserted into an expression vector which is transfected into a suitable host. The DNA is expressed and the protein is harvested. See Sambrook et al., Id.

Equivalents of the mutant or wild-type p53 protein may also be used in the vaccine of the invention. Such equivalents include analogs that induce an immune response comparable to that of the mutant or wild-type p53 protein. In addition, such equivalents are immunologically cross-reactive with their corresponding mutant or wild-type p53 protein. The equivalent may, for example, be a fragment of the protein, or a substitution, addition or deletion mutant of the mutant or wild-type p53 protein.

The mutant or wild-type p53 protein fragment preferably contains sufficient amino acid residues to define an epitope of the antigen. The fragment may, for example, be a minigene encoding only the epitope. Methods for isolating and identifying immunogenic fragments from known immunogenic proteins are described by Salfeld et al. in J. Virol. 63, 798-808 (1989) and by Isola et al. in J. Virol. 63, 2325-2334 (1989).

The wild-type or mutant p53 protein fragments may be expressed by truncated wild-type or mutant p53 genes. The p53 gene is composed of 11 exons. The first exon (213 bp) is non-coding and is located 8-10 Kb away from the second exon which contains the translational start codon. In the present invention, the truncated p53 genes encoding wild-type or mutant p53 protein fragments may lack any one exon or more than one exon, or any portion thereof. The number of exons lacking from the truncated p53 genes encoding wild-type or mutant p53 protein fragments preferably lack 2-4 exons, and more preferably, lack any of the first 4 exons. In another preferred embodiment, the truncated p53 genes lack all first 4 exons, and thereby comprise exons 5-11.

If the fragment defines a suitable epitope, but is too short to be immunogenic, it may be conjugated to a carrier molecule. Some suitable carrier molecules include keyhole limpet hemocyanin, Ig sequences, TrpE, and human or bovine serum albumen. Conjugation may be carried out by methods known in the art. One such method is to combine a cysteine residue of the fragment with a cysteine residue on the carrier molecule.

Equivalent proteins have equivalent amino acid sequences. An amino acid sequence that is substantially the same as another sequence, but that differs from the other sequence by one or more substitutions, additions and/or deletions, is considered to be an equivalent sequence. Preferably, less than 25%, more preferably less than 10%, and most preferably less than 5% of the number of amino acid residues in a sequence are substituted for, added to, or deleted from the proteins of the invention.

For example, it is known to substitute amino acids in a sequence with equivalent amino acids. Groups of amino acids generally considered to be equivalent are:

(a) Ala(A) Ser(S) Thr(T) Pro(P) Gly(G);

(b) Asn(N) Asp(D) Glu(E) Gln(Q);

(c) His(H) Arg(R) Lys(K);

(d) Met(M) Leu(L) Ile(I) Val(V); and

(e) Phe(F) Tyr(Y) Trp(W).

The mutant or wild-type p53 protein of the invention unexpectedly induces an effective immune response when properly presented to the immune system. For the purposes of this specification, an effective immune response inhibits, i.e. prevents, slows or stops, the growth of cancer cells, or eliminates cancer cells. The effective immune response is preferably a killer T-cell response. The mammal may be a human or animal typically used for experimentation, such as mice, rats or rabbits.

The mutant or wild-type p53 is presented to the immune system as a vaccine by a vehicle. For example, the mutant or wild-type p53 may be present on the surface of an antigen presenting cell or combined with a pharmaceutically acceptable adjuvant.

Antigen presenting cells are generally eukaryotc cells with major histocompatibility complex (MHC), preferably Class II, gene products at their cell surface. For the purposes of this specification, antigen presenting cells also include recombinant eucaryotc cells such as peripheral blood cells and recombinant bacterial cells. Some examples of antigen presenting cells as defined by this specification include dendritic cells, macrophages that are preferably MHC Class II positive, monocytes that are preferably MHC Class II positive, and lymphocytes.

In one embodiment of the subject invention, the antigen presenting cell is a recombinant eucaryotc cell that expresses exogenous DNA encoding mutant or wild-type p53 protein. The recombinant eucaryotc cell may be prepared in vivo or in vitro.

Suitable cloning/expression vectors for inserting DNA into eucaryotic cells include well-known derivatives of SV-40, adenovirus, cytomegalovirus (CMV), and retrovirus-derived DNA sequences. Any such vectors, when coupled with vectors derived from a combination of plasmids and phage DNA, i.e. shuttle vectors, allow for the cloning and/or expression of protein coding sequences in both procaryotc and eucaryotic cells.

Other eucaryotic expression vectors are known in the art, e.g., P. J. Southern and P. Berg, J. Mol. Appl. Genet 1, 327-341 (1982); S. Subramani et al, Mol. Cell. Biol. 1, 854-864 (1981); R. J. Kaufmann and P. A. Sharp, “Amplification And Expression Of Sequences Cotransfected with A Modular Dihydrofolate Reductase Complementary DNA Gene,” J. Mol. Biol. 159, 601-621 (1982); R. J. Kaufmann and P. A. Sharp, Mol. Cell. Biol. 159, 601-664 (1982); S. I. Scahill et al, “Expression and Characterization of the Product of a Human immune Interferon DNA Gene in Chinese Hamster Ovary Cells,” Proc. Natl. Acad. Sci. USA 80, 4654-4659 (1983); G. Urlaub and L. A. Chasin, Proc. Natl. Acad. Sci. USA 77, 4216-4220, (1980).

In one embodiment, DNA encoding mutant or wild-type p53 is inserted into the eucaryotic cell in vivo using recombinant viral vectors. These vectors include an attenuated recombinant poxvirus, such as vaccinia virus, for example, parrot pox, that has its nonessential virus-encoded genetic functions inactivated. Other examples of suitable vaccinia viruses include the Copenhagen vaccine strain of vaccinia virus called NYVAC, or the avipoxvirus genus canarypox virus (ALVAC), which are described in International Application Number PCT/US92101906, filed Mar. 2, 1992, U.S. Pat. No. 5,364,773, issued Nov. 15, 1994, and International Application No. PCT/US94/00888, filed Jan. 21, 1994. Techniques for the insertion of foreign DNA into a viral genome such as the vaccinia genome are known in the art (see PCT/US92/01906). Plasmid vectors for the construction of recombinant viruses are described in, for example, Chakrabarti et al. (1985) Mol. Cell Biol. 5:3403; Mackett et al., (1984) J. Virol. 49:857; and Moss (1987), page 10 of Gene Transfer Vectors for Mammalian Cells, Miller and Calos, eds., Cold Spring Harbor Laboratory, Cold Spring Harbor, N.Y. Expression of the mutant or wild-type p53 protein then occurs in vivo in an antigen presenting cell in subjects immunized with the recombinant poxvirus.

In another embodiment, DNA encoding mutant or wild-type p53 is inserted into the eucaryotic cell in vitro using known techniques, such as the retroviral transduction techniques described for tumor infiltrating lymphocytes (TILs) (S. A. Rosenberg et al., NEJM, 323(9):570-578 (Aug. 30, 1990) and K. Culver et al., PNAS USA 88:3155-3159 (April 1991)).

In another embodiment, minigenes encoding the mutant or wild-type p53 epitope are inserted into the eucaryotic cell in vitro using known techniques (see Hahn et al. Proc. Natl. Acad. Sci. USA 89:2679-2683 (April 1992).

The mutant or wild-type p53 protein may also be presented to the immune system on the surface of recombinant bacterial cells. A suitable recombinant bacterial cell is an avirulent strain of Mycobacterium bovis, such as bacille Calmette-Guerin (BCG), or an avirulent strain of Salmonella, such as S. typhimurium. The recombinant bacterial cells may be prepared by cloning DNA comprising the active portion of the p53 protein in an avirulent strain, as is known in the art; see, for example, Curtiss et al., Vaccine 6, 155-160 (1988) and Galan et al., Gene 94, 29-35 (1990) for preparing recombinant Salmonella and Stover, C. K. et al., Vaccines 91, Cold Spring Harbor Laboratory Press, pp. 393-398 (1991) for preparing recombinant BCG.

Cloning vectors may comprise segments of chromosomal non-chromosomal and synthetic DNA sequences. Some suitable prokaryotic cloning vectors include plasmids from E. coli, such as colE1, pCR1, pBR322, pMB9, pUC, pKSM, and RP4. Prokaryotic vectors also include derivatives of phage DNA such as M13, fd, and other filamentous single-stranded DNA phages.

Vectors for expressing proteins in bacteria, especially E. coli, are also known. Such vectors include the pK233 (or any of the tac family of plasmids), T7, and lambda P_{L}. Examples of vectors that express fusion proteins are PATH vectors described by Dieckmann and Tzagoloff in J. Biol. Chem. 260, 1513-1520 (1985). These vectors contain DNA sequences that encode anthranilate synthetase (TrpE) followed by a polylinker at the carboxy terminus. Other expression vector systems are based on beta-galactosidase (pEX); lambda P_{L}; maltose binding protein (pMAL); glutathione S-transferase (pGST)—see Gene 67, 31 (1988) and Peptide Research 3, 167 (1990).

The expression vectors useful in the present invention contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, major operator and promoter regions of phage lambda, the control region of fd coat protein, and promoters derived from polyoma, adenovirus, retrovirus, and simian virus, e.g., the early and late promoters of SV40, and other sequences known to control the expression of genes in prokaryotic or eukaryotc cells and their viruses or combinations thereof.

As shown in Examples 2-3, immunization with recombinant BCG or recombinant ALVAC vaccines expressing p53 peptide sequences protected mice against challenge with a p53 expressing tumor cell line.

The vaccine may further comprise pharmaceutically acceptable adjuvants, such as muramyl peptides, lymphokines, such as interferon, interleukin-1 and interleukin-6, or bacterial adjuvants. The adjuvant may comprise suitable particles onto which the mutant or wild-type p53 protein is adsorbed, such as aluminum oxide particles. These vaccine compositions containing adjuvants may be prepared as is known in the art.

An example of a bacterial adjuvant is BCG. When used as an antigen presenting cell as described above, recombinant BCG may additionally act as its own adjuvant. In this case, additional adjuvant may not be needed although one or more additional adjuvants may optionally be present. When used in its natural (non-recombinant) state, BCG acts solely as an adjuvant by being combined with mutant or wild-type p53, resulting in a form that induces an effective immune response.

The vaccine may also comprise a suitable medium. Suitable media include pharmaceutically acceptable carriers, such as phosphate buffered saline solution, liposomes and emulsions.

The invention further includes a method of inhibiting the growth of tumors in mammals comprising treating a mammal having a tumor or at imminent risk of obtaining a tumor with an immunologically effective amount of a vaccine comprising mutant or wild-type p53. A mammal is at imminent risk of obtaining a tumor if the mammal is diagnosed as having an abnormal, precancerous condition.

The mutant or wild-type p53 is presented to the immune system of the mammal in a form that induces an effective immune response, i.e., either on the surface of an antigen presenting cell or combined with a pharmaceutically acceptable adjuvant. The mutant or wild-type p53 is preferably in a medium such as a pharmaceutically acceptable carrier.

The vaccine may be administered to a mammal by methods known in the art. Such methods include, for example, oral, intravenous, intraperitoneal, subcutaneous, intramuscular, topical, or intradermal administration.

The following Example section is set forth to aid in an understanding of the invention. This section is not intended to, and should not be construed to, limit in any way the invention as set forth the claims which follow thereafter.

### EXAMPLES

### Example 1

### Immunization with p53-Expressing Tumor Cells

The following experiment demonstrates that mutant p53 expression in p53 negative tumor cells can lead to rejection of these cells. A murine model system is described in which the host immune system was activated by challenge with mutant p53 expressing tumor cells. This activation resulted in 1) spontaneous regression of mutant p53 expressing tumors, 2) rejection of tumors caused by a highly tumorigenic variant cell line after immunization with p53 containing cells. 3) induction of a cellular and humoral immune response directed against p53 and 4) increased susceptibility to p53 induced tumors of mice carrying a p53 transgene. Together these findings that p53 can function as a tumor specific rejection antigen (TSRA) in vivo establish a murine model system for the development of human cancer vaccines aimed at the mutant p53 protein.

### Example 1A

### Materials and Methods

The (10)3 cell line is described in Harvey and Levine (1991) Genes Dev 5, 2375-2385. The (10)3 cell lines expressing mutant p53 (e.g. (10)3-273.1) are described in Dittmer, et al. (1993) Nature Genetics 4,42-46. Cell lines bearing an NT designation (e.g. (10)3-273.1NT24) are derived from nude mouse tumors which formed after injection of 5*10⁶ cells of the respective cell line under the skin of nude Balb c/J mice. The (10)3/Tx4BT87 cell line was derived from a tumor caused by injecting 10⁷ cells of a spontaneous foci of (10)3 cells under the skin of Balb c/J mice. This cell line does not express p53 protein.

For the analysis of MHC-I expression, fibroblast cells were scraped from the dish and resuspended in DMEM/0.5% calf serum (CS). Spleen cells and suspension cells were resuspended in DMEM/0.5% CS. 106 cells were pelleted and incubated with the primary antibody in 100 III DMEM/1% BSA for 1 h at 4° C. in the dark. 20-1 l anti-H2-K^{d} monoclonal supernatant and 2 III polyclonal ant-H2-K^{b} serum were used. The cells were washed three times with PBS/1% BSA, pelleted and incubated for ½ h with 2 μl phycoerythrin-conjugated anti-mouse-IgG (Cappel) in 100 μl DMEM, 0.5% CS. The cells were washed three times with PBS/1% BSA and were analyzed using a FACS analyzer (Counter).

For the analysis of serum antibody levels, mice were bled and the blood was left at 40° C. ON. The blood-clot was pelleted and the serum stored at −80° C. For analysis, 5 μl serum were diluted into 50 μl PBS and 10 μl of this mix were incubated ON at 4° C. with 500 μl [35S]-methionine labeled cell-extract from SV80 cells, which are SV40 transformed human fibroblasts and which express high levels of p53 and SV40 T antigen, in cytoplasmic lysis buffer (CLB) (10 mM Tris pH 7.4, 250 mM sucrose, 160 mM KCl, 50 mM ε-amino-caproic acid, 0.5% NP-40 supplemented to 3 mM 13-mercaptoethanol, 1 mM PMSF and 0.28 TIU/ml aprotinin immediately prior to use) or 500 μl in vitro translated [35S]-methionine labeled human p53 in CLB-buffer and 25 μl proteinA sepharose (stock solution: 50% w/w proteinA sepharose, Pharmacia in 50 mM Tris pH 7.4, 5 mM EDTA, 0.5% NP-40, 150 mM NaCl). The proteinA sepharose beads were washed three times in SNNTE (50 mM Tris pH 7.4, 5 mM EDTA, 5% sucrose, 1% NP-40, 0.5 M NaCl) and analyzed by 8% SDS-PAGE (Laemmli (1970) Nature 27, 690-). After completion of the run the gel was fixed in methanovacetic acid for 30 min and washed twice 10 min in dH2O and once in 1 M sodium salicylate for ½ hour.

Total spleen cells were used as effector cells in cellular cytotoxicity assays. Spleens were removed into 5 ml DMEM/10% FCS 11-20 days after priming and dissociated by gentle grinding between the frosted ends of glass slides. The cells were pelleted by centrifugation at 500 g for 10 minutes and resuspended in 4 ml 0.95% (w/v) ammonium chloride. Cells were left on ice for 5 minutes to lyse erythrocytes. After that 6 ml 2×DMEM was added and the cells were immediately pelleted. The cells were resuspended in 5 ml DMEM/10% FCS, counted and if necessary further purified over a ficoll (Pharmacia) gradient according to manufacturer's recommendation. Cells were resuspended at 10⁷ cells per ml in DMEM/10% FCS and cultured for 24 h at 37° C. The cells were than co-cultured for 5 days with 2'10⁵ mitomycin C treated stimulator cells per ml (50:1). Stimulator cells were incubated for 2 h with 10 ml 15 ug/ml mitomycin C (Sigma) and washed 3 times in DMEM/10% FCS. Where indicated human IL-2 (Gibco) was added at 250 U/ml. For the [51Cr]-release assay the target cells were trypsinized and resuspended at 1*10⁶ cells per ml and labeled with 300 μCi [⁵¹Cr] (NEN) per ml for 2 hours at 37° C. The cells were washed four times in DMEM/10% FCS, resuspended at 10⁵ cells per ml and dispersed at 100 μl per well into a 96 well plate (Coming). Spleen cells were resuspended at 10⁷ cells per ml in DMEM/10% FCS and diluted to give the respected effector to target cell ratio. 100 μl effector cells were added to 100 μl target cells and incubated for six hours at 37° C. Before the incubation period the 96 well plates were centrifuged at 500 g for 5 min to allow the cells to contact each other. After the incubation period the 96 well plates were centrifuged at 1000 g for 5 min and 100 μl supernatant was counted in a Beckman gamma counter. Maximal release was determined by adding 100 μl 5% SDS. Spontaneous release was determined by adding 100 μl DMEM. Only experiments in which spontaneous lysis was less than 20% of the maximal release were included in the analysis. All experiments were performed in quadruplicates. Percent lysis was calculated as follows:

$$\frac{c . p . m in presence of Tc - spontaneous release}{maximal release - spontaneous release} * 100$$

Balb c/J mice were purchased from Jackson laboratory, Maine, and housed under P3 conditions. The results are presented as the number of mice with tumors over the total number of mice injected. Tumors grew progressively until they were 0.5-1.0 cm in size and became necrotic. The animals were sacrificed by cervical dislocation at that time. Animals displaying no tumor after 3 standard deviations (SD) of the control group were considered tumor-free (Heitjan, (1993) Cancer Res 53, 6042-6050). Tumor diameter was measured at weekly or biweekly intervals. All graphs of tumor growth plot the tumor diameter (d) in mm over time in days. This was fitted to an exponential function d(t)=tₒ*exp(K*t) least square approximation using Cricketgraph™. tₒ represents the time at which a tumor was initially visible, i.e. d(tₒ)≦1 mm. t_{1/2}=1n2/K is called the doubling time. It is valid to display the diameter d=2*r rather than tumor volume V if we assume that for solid tumors, active tumor growth is limited to the outermost cells and thus r is exponentially dependent on time t (Edelstein-Keshet, (1987) Mathematical Models in Biology (New York: McGraw Hill)).

### Example 1B

### Tumorigenicity of (10)3 Mutant p53 Cells in Immunocompetent Mice

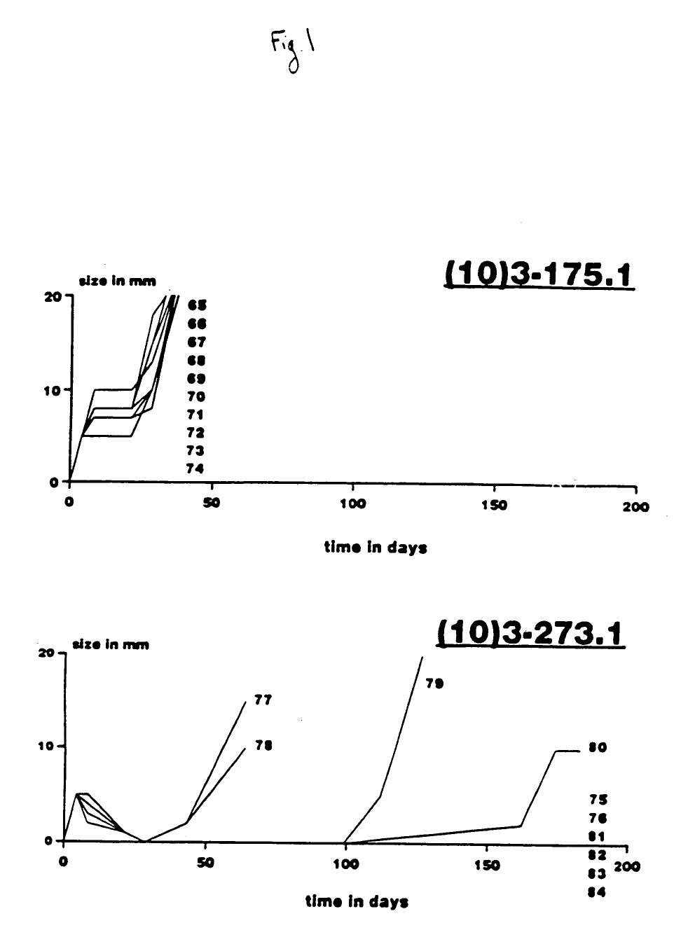

(10)3 cells are spontaneously immortalized fibroblasts which were derived from Balb c/J mice (Harvey, (1991) Genes Dev. 5, 2375-2385). The cells are devoid of endogenous p53 expression. Upon expression of human or mouse mutant p53 protein the cells acquire the ability to form tumors in immunodeficient Balb c/J nu/nu mice (Dittmer, et al. (1993) Nature Genetics 4, 42-46). However, the majority of cell lines expressing mutant p53 alleles do not form tumors in immune-competent syngeneic Balb c/J mice (Table 1). The cell lines designated (10)3-248.1, (10)3-175.1 and (10)3-273.1 are exceptions because they form tumors when injected subcutaneously (s.c.) into Balb c/J mice at 1*10⁷ cells per animal. All cell lines were generated by transfection of plasmids which contain in cis the gene for the respective human mutant p53 under control of the human cytomegalovirus (CMV) promoter/enhancer and the gene coding for resistance to G418 (Dittmer, et al. (1993) Nature Genetics 4, 42-46). (10)3-175.1 and (10)3-273.1 cell lines express high levels of human p53 protein mutated at amino acid 175 and at amino acid 273 respectively. However, the growth characteristics of (10)3-175.1 and (10)3-273.1 in immunocompetent Balb c/J mice are quite different: (10)3-175.1 induced tumors grew progressively following a lag period of 20 days, whereas (10)3-273.1 induced tumors appeared as early as (10)3-175.1 induced tumors, but these tumors disappeared and only much later did a subset (4/10) of the mice develop tumors (FIG. 1). Both (10)3-175.1 and (10)3-273.1 induced tumors exhibited undifferentiated neoplastic morphology typically seen in tumors induced by s.c. injection of tumor cells. The tumor cells continue to express human p53 as demonstrated by immunohistochemistry of tumor sections using a human p53-specific antibody (mab1801) and immunoblot analysis. Cell lines established from these tumors continued to express human p53 as demonstrated by immunoprecipitation of metabolically labeled cell extract using mab 1801 and were resistant to G418. This behavior is consistent with the notion that tumorigenicity of (10)3 cells is dependent upon continuous expression of the mutant p53 protein.

Table 1:

The mutant p53 expressing cell lines are described in Dittmer, et al., 1993. Tumorigenicity in either Balb c/J nu/nu or Balb c/J mice is given by the number of mice with tumors over the total number of mice inoculated with 5*10⁶ cells/mouse or 1*10⁷ cells per mouse, respectively.

TABLE 1

| | | tumorigenicity | | | |
|---|---|---|---|---|---|
| | | nu/nu | balb c/J | | |
| p53 allele | cell line | incidence | % | incidence | % |
| parental | | | | | |
| none | (10)3 | 0/10 | 0 | 0/10 | 0 |
| spontaneous | | | | | |
| foci | | | | | |
| none | (10)3/Tx4 | — | | 1/5 | 30 |
| | (10)3/Tx5 | — | | 2/5 | |
| human | | | | | |
| mutant | | | | | |
| 175 R to H | (10)3/175.1 | 3/3 | 77 | 10/10 | 31 |
| | (10)3/175.2 | 3/3 | | 0/5 | |
| | (10)3/175.3 | 1/3 | | 0/5 | |
| 248 R to W | (10)3/248.1 | 3/3 | | 3/3 | |
| | (10)3/248.2 | 1/3 | | 0/3 | |
| 271 R to H | (10)3/273.1 | 3/3 | | 2/10 | |
| 281 D to G | (10)3/281.1 | 3/3 | | 0/3 | |
| mouse | | | | | |
| mutant | | | | | |
| KH215 | (10)3KH215.1 | 1/3 | | 0/5 | |
| | (10)3KH215.2 | 5/6 | | 0/5 | |
| nude mouse | | | | | |
| tumor derived | | | | | |
| KH215 | (10)3/KH215NT140 | — | | 2/3 | 89 |
| 175 R to H | (10)3/175.1NT20 | — | | 5/5 | |
| 273 R to H | (10)3/273.1NT24 | — | | 5/5 | |
| 248 R to H | (10)3/248.1NT164 | — | | 2/3 | |
| | (10)3/248.2NT167 | — | | 3/3 | |
| nude mouse | | | | | |
| tumor derived | | | | | |
| p53 negative | | | | | |
| none | (10)3/Tx4BT87 | — | | 3/3 | 100 |

### Example 1C

### Antibody Response to p53 Positive Tumor Cells

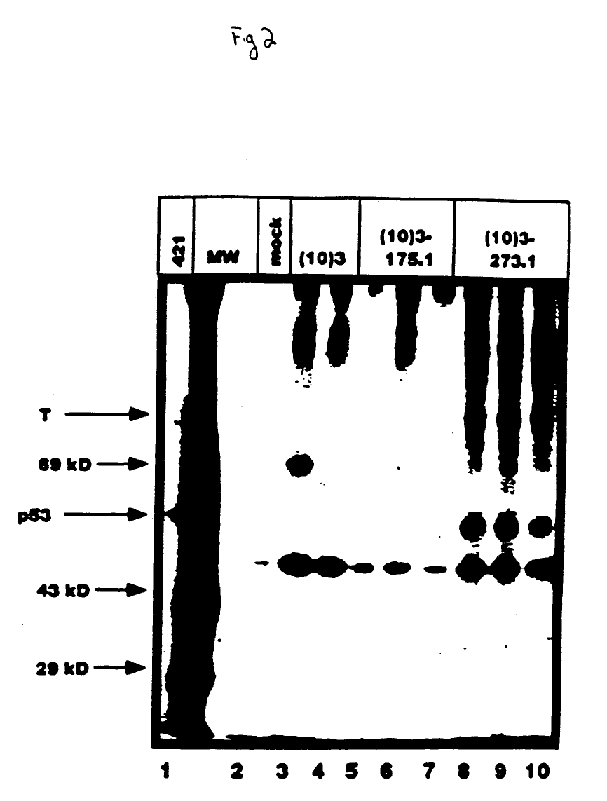

Remission of (10)3-273.1 induced tumors coincided with the appearance of p53-specific antibodies. In contrast mice which had been injected with the parental (10)3 cells, (10)3-175.1 cells or untreated animals were devoid of anti-p53 antibodies (FIG. 2). The antibody response was measured by ELISA and immunoprecipitation using 3 μl of whole mouse serum to precipitate human wild-type p53 from SV40 transformed human SV80 cell extracts when bound to protein A sepharose beads (FIG. 2). These experiments demonstrate that (10)3-273.1 cells are inherently immunogenic whereas (10)3-175.1 cells which also express p53 are not. These cell line specific differences were exploited to study a possible involvement of p53 as one of the TSRAs.

### Example 1D

### Tumor Protection by a (10)3 Mutant pS3 Cell Line

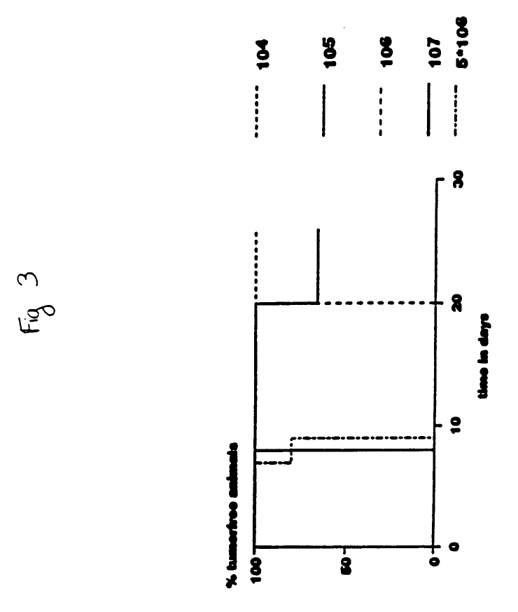

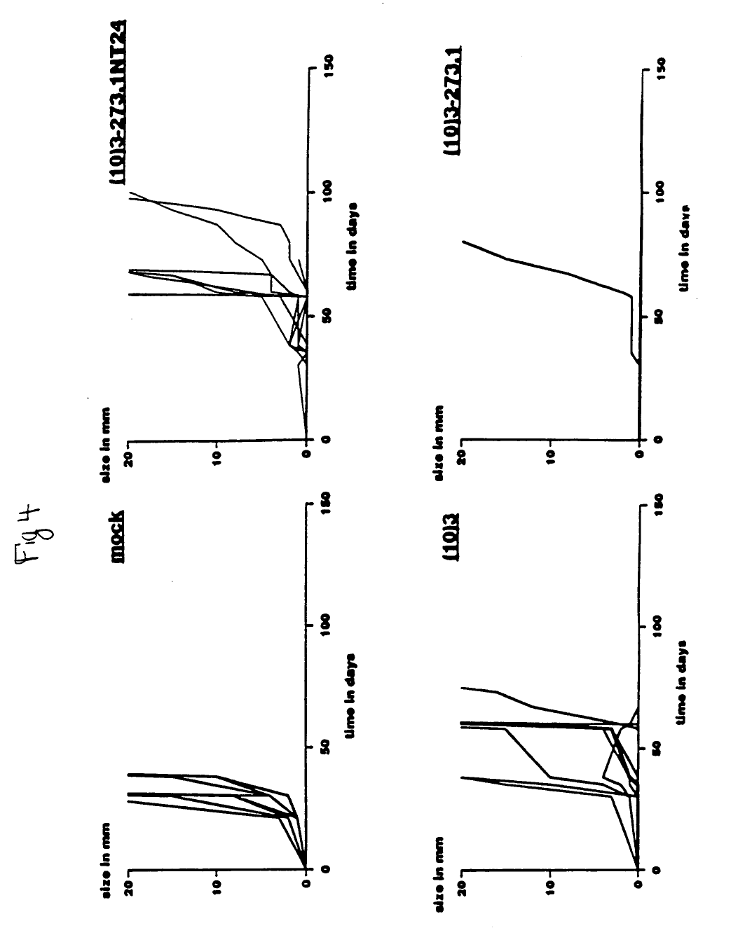

To investigate whether the regression of (10)3-273.1 induced tumors was due to systemic immunity, we asked whether immunization with (10)3-273.1 cells could protect animals from challenge with a highly tumorigenic clone: (10)3-273.1NT24. (10)3-273.1 NT24 cells are derived from a tumor induced by injection of (10)3-273.1 cells into a nude mouse. They express mutant p53 protein at levels comparable to those seen in the (10)3-273.1 cells. Unlike the parental (10)3-273.1 cells but like other mutant p53 positive (10)3 derivatives passaged in nude mice (Table 1), (10)3-273.1 NT24 cells form progressively growing tumors in Balb c/J mice (FIGS. 3 and 4). Challenge with 1*10⁵ (10)3-273.1NT24 cells has been used to study immune protection by mitomycin C treated (10)3-273.1 cells (FIG. 4). In one particular experiment (experiment #2), 10 out of 10 untreated control mice developed progressively growing tumors at 21 days post challenge. Immunization with the parental (10)3 cells delayed tumor growth, but nevertheless 8 out of 10 mice succumbed to tumors (40+12 days post challenge). Immunization with (10)3-273.1 protected against challenge with (10)3-273.1NT24 cells. 9 out of 10 remained tumor-free at 145 days post challenge. Immunization with (10)3-273.NT24 cells also delayed tumor onset, but could not protect against self challenge. 9 out of 10 animals succumbed to tumors at 54+15 days post challenge. The tumor challenge experiments have been repeated three times with a total of 25 animals for each group (Table 2). At a higher challenge dose of 5*10⁶ cells per animal both immunized and non-immunized animals develop tumors (experiment #3, Table 2). Here, immunization with (10)3-273.1 cells delayed tumor onset (0.01<p<0.05 for immunization with (10)3-273.1 cells versus non-immunized mice using the Mann-Whitney Rank Sum Test). Immunization with (10)3 cells did not result in any significant delay of tumor onset.

TABLE 2
The mice were injected twice s.c. with 1 * 10⁶ mitomycin C
treated cells one week apart and challenged one week later with
the indicated dose of (10)3-273.1NT24 cells. Tumorigenicity in
Balb c/J mice is given by the number of mice with tumors
over the total number of mice inoculated. Day of first tumor
appearance (≧1 mm diameter) ± standard deviation
mean (SD) is given for each entry.

| | | |
|---|---|---|
| immunized with | tumorigenicity | time in days ± SD |

experiment 1: challenge with 1 * 10⁵ (10)3-273.1NT24 cells

| | | |
|---|---|---|
| mock | 5/5 | 69 ± 18 |
| (10)3 | 4/5 | 115 ± 56 |
| (10)3-273.1 | 0/5 | >250 |
| (10)3-273.1NT24 | 3/5 | 147 ± 16 |

experiment 2: challenge with 1 * 10⁵ (10)3-273.1NT24 cells

| | | |
|---|---|---|
| mock | 10/10 | 21 ± 0 |
| (10)3 | 8/10 | 40 ± 12 |
| (10)3-273.1 | 1/10 | 35; > 145 |
| (10)3-273.1NT24 | 9/10 | 54 ± 15 |

experiment 3: challenge 5 * 10⁶ (10)3-273.1NT24 cells

| | | |
|---|---|---|
| mock | 5/5 | 8 ± 1 |
| (10)3 | 10/10 | 9 ± 4 |
| (10)3-273.1 | 10/10 | 13 ± 4 |
| (10)3-273.1NT24 | 5/5 | 17 |

### Example 1E

### Efficiency and Specificity of Protection

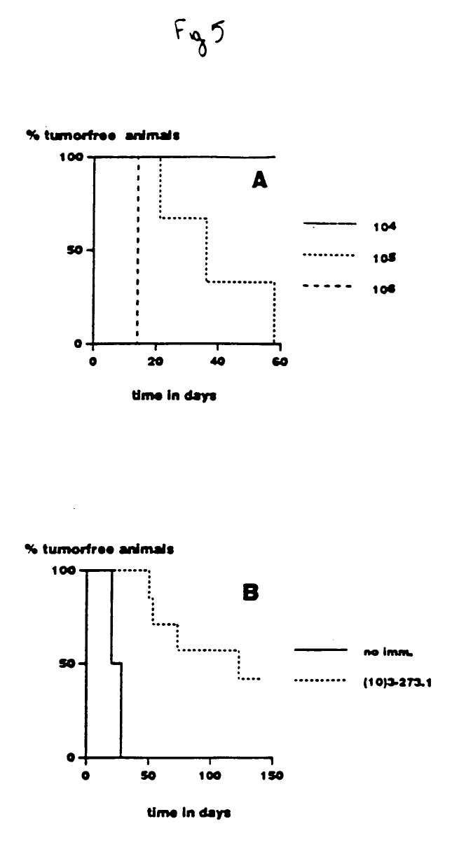

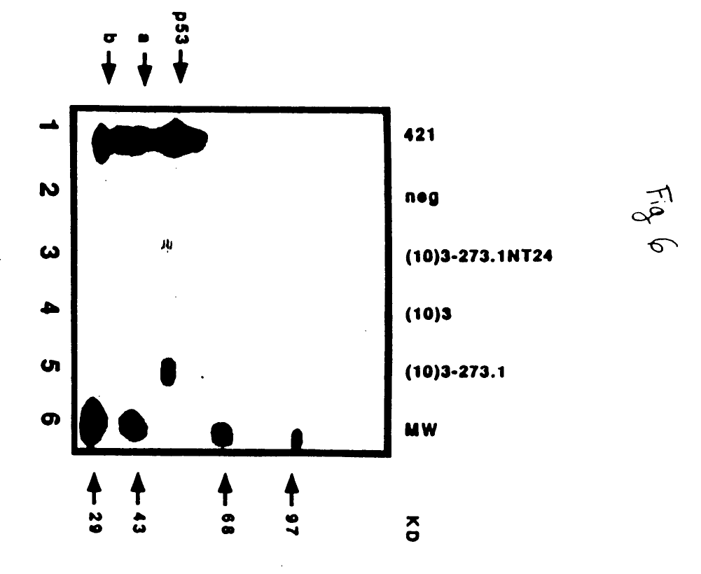

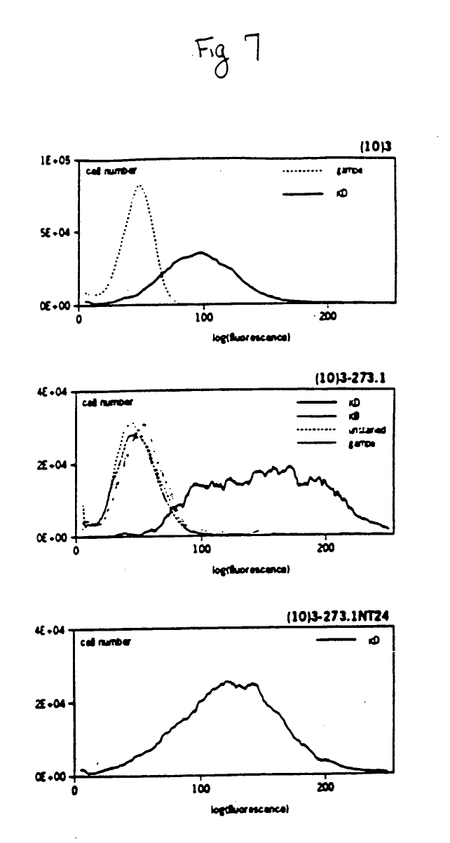

In order to address the question whether the protective effect seen after immunization with (10)3-273.1 cells was directed against mutant p53 or mutant p53 dependent TSRAS, (10)3-273.1 immunized animals were challenged with a tumorigenic clone of (10)3 cells (10)3-tx4BT87 which does not express p53. (10)3-tx4BT87 is a cell line derived from a tumor induced by spontaneously transformed (10)3 cells in Balb c/J mice. It forms progressive tumors in Balb c/J mice (FIG. 5A). Immunization with (10)3-273.1 cells delayed tumor onset but protected only a subset of animals (3/7) (FIG. 5B). Immunization with (10)3-273.1 cells did not protect against a p53 negative cell line (10)3-tx4BT87 as effectively as against the mutant p53 positive (10)3-273.INT24 cells. As seen after injection of live (10)3-273.1 cells (FIG. 2), the mitomycin C treated (10)3-273.1 cells were also able to elicit an anti-p53 IgG response. This was measured by ELISA and the ability of pooled serum samples taken at the time of tumor challenge to precipitate in vitro translated human p53. No anti-p53 antibodies could be detected in mice which were either non-immunized or mice immunized with (10)3 or (10)3-273.1NT24 cells (FIG. 6). The difference in protection efficiency in experiment #2 (Table 2) of (10)3-273.1 NT24 versus (10)3-273.1 can be reconciled in light of the fact that (10)3-273.1 cells are much more effective in evoking an immune response than the (10)3-273.1NT24 subclone. Both cells express similar levels of H2-K^{d} (Table 3 and FIG. 7).

TABLE 3
FACS analysis of MHC-I expression in the cell lines used
in this study. Phycoerythrin conjugated goat anti-mouse
antibody (PE, Molecular Probes) was used at 1:100
dilution. 2 μl anti-K^{b} ascites fluid (S. Tevethia)
or 20 μl anti-K^{d} hybridoma SN (L. Sherman)
was used per 10⁶ cells. Shown are mean channel
of log relative fluorescence ± standard deviation (SD)
and the percent of cells shifted.

| cell line | antibody | log (fluorescene) | % cells |
|---|---|---|---|
| (10)3 | GAMPE | 3 ± 2 | 93 |
| | anti-K^{D} | 87 ± 73 | 75 |
| (10)3-273.1 | GAMPE | 3 ± 2 | 91 |
| | anti-K^{B} | 3 ± 2 | 93 |
| | anti-K^{D} | 116 ± 91 | 95 |
| P815 | GAMPE | 5 ± 3 | 90 |
| | anti-K^{D} | 140 ± 93 | 90 |
| P815:273 | GAMPE | 2 ± 2 | 98 |
| | anti-K^{D} | 303 ± 194 | 96 |

### Example 1F

### Cellular Cytotoxicity

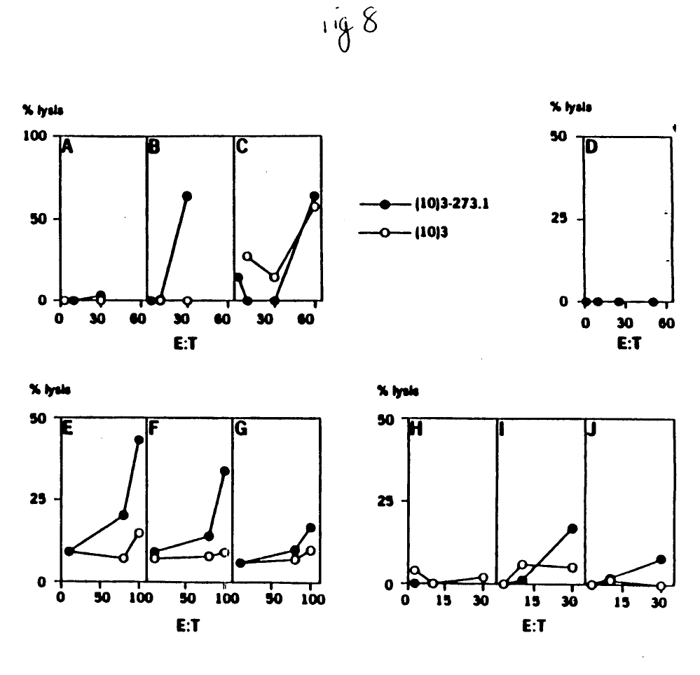

Tumor rejection is predominately mediated by cytotoxic lymphocytes. Therefore, the presence of cellular cytotoxicity directed towards p53 was analyzed. Balb c/J mice were injected with (10)3-273.1 cells as before and splenic lymphocytes were isolated over a ficoll gradient 8 to 9 weeks later. A typical isolation yielded 10⁷ cells per spleen (>90% lymphocytes as determined by light scatter of which 10-20% were CD4 and 10-20% CD8 single positive cells). 10⁶ lymphocytes per ml were co-cultured in DMEM/10% FCS with mitomycin C treated (10)3 or (10)3-273.1 stimulator cells at a effector to stimulator ratio of 50:1 for 3 to 5 days at 37° C., 5% CO₂. No exogenous IL-2 was added to the cultures. Lymphocytes proliferated in the presence of either (10)3 or (10)3-273.1 stimulator cells but not when cultured without stimulator cells. They were tested for cellular cytotoxicity in 4 h [⁵¹]-Cr release assays. In two separate experiments with three mice each lymphocytes from mice immunized with (10)3-273.1 cells and stimulated with (10)3-273.1 cells in vitro were able to kill (10)3-273.1 cells but not (10)3 cells (FIG. 8, e-j). Lymphocytes of non-immunized animals but stimulated with (10)3-273.1 cells in vitro showed no killing of either (10)3 nor (10)3-273.1 cells (FIG. 8, d) even when cultured in the presence of stimulator cells and 350 U/ml rhIL-2 (FIG. 8, a). Co-culture of lymphocytes from a mouse immunized with (10)3-273.1 cells with IL-2 and (10)3-273.1 resulted in killing of both (10)3 and (10)3-273.1 target cells (FIGS. 8b,c). However, this was not a consistent observation. All target cells express the respective H2-K^{d} allele (Table 3 and FIG. 7).

### Example 1G

### Susceptibility of Transgenic Mice Expressing Human Mutant p53 to Mutant p53-Expressing Tumors

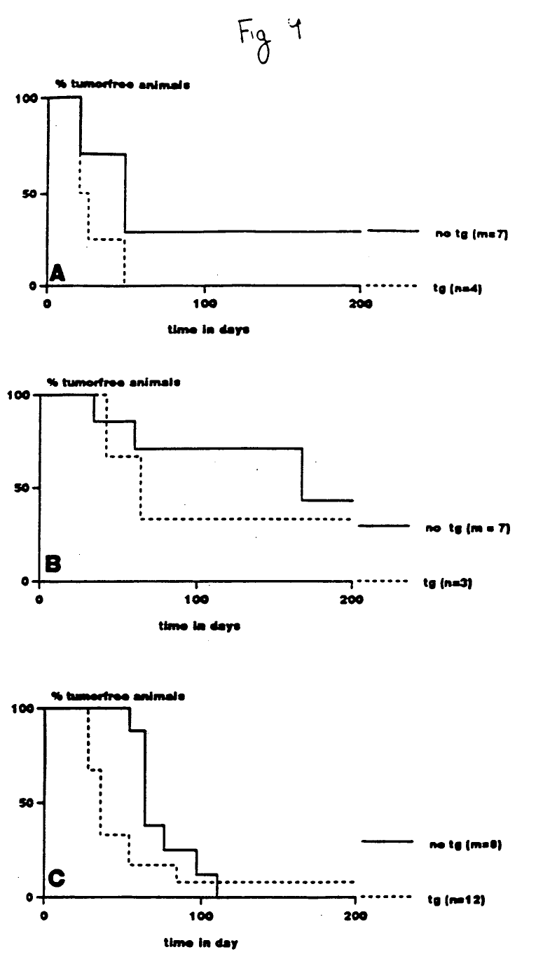

To further establish p53 as the dominant TSRA in (10)3-273.1 cells, we studied the susceptibility to (10)3-273.1 induced tumors in mice expressing human p53 transgenes. Male mice which were heterozygous for human p53 mutant 175 or 273 transgene were crossed with Balb clJ females and their offspring was injected s.c. with 1*10⁶ (10)3-273.1 cells per animal. Mice carrying the p53 transgene developed progressive growing tumors whereas their non transgenic littermates did not develop tumors or exhibited a delayed tumor onset (FIG. 9). Cell lines established from these tumors expressed human p53 protein. The observed differences in tumor onset were significant to ps0.001 in a stratified Wilcoxon-Test. The differences are less pronounced compared to the challenge experiments in inbred Balb c/J mice which might be due to increased individual genetic variation between these DBAxC57BI6xBalbc/J animals. The increased tumor susceptibility of the transgenic mice demonstrates that the p53 transgenic mice are tolerant to human p53 and thus are impaired in their ability to mount an effective immune response to tumor cells which express the human mutant p53 whereas their siblings react similarly to normal Balb c/J mice.

### Example 2

### Immunization with p53-Expressing BCG Bacteria

The following table (Table 4) shows results concerning the ability to protect mice from tumors by immunizing them with human p53 protein expressed in BCG bacteria. Commercially available Pasteur and Connaught (Ontario, Canada) BCG strains were used. The p53 gene is inserted into the BCG bacteria by known methods (see Snapper, S. B. et al., Proc. Natl. Acad. Sci. USA 85:6987-6991 (1988); Stover, C. K. et al., Vaccines 91, Cold Spring Harbor Laboratory Press, pp. 393-398 (1991); Stover, C. K. et al., Nature 351:456-460 (1991), Kalpana, B. V. et al., Proc. Natl. Acad. Sci. USA 88:5433-5437 (1991): and published International Applications Nos. WO 8806626, Sep. 9, 1988; WO 9000594. Jan. 25, 1990; and WO 9222326, Dec. 23, 1992).

As is shown in Table 4, the Connaught strain of BCG expressing human p53 exons 5-11 at high level will protect against isogenic tumorigenic cells (see experiments #1a, #1b, and #1c, in which only two out of fifteen mice formed tumors). The BCG bacteria alone, i.e. containing a vector but without p53 (experiment #2a) or not injecting BCG bacteria at all into the mice (experiments #2b, #2c) did not protect mice (21/25 mice formed tumors). The Pasteur strain expressing full length p53 at low levels was less effective than the Connaught strain expressing truncated p53 in protecting mice from the challenge as shown in experiments #3a, #3b, #3c (19120 mice formed tumors, some of which progressed more slowly than the tumors formed in the mice in the control experiments (#2a-c)).

TABLE 4
Experiments Employing BCG-p53 to Protect
Against Tumors Containing p53 Proteins

| Number of | | | | challenge of tumor cells^{d} | | |
|---|---|---|---|---|---|---|
| colony forming | | | booster | # tumors/ | | time for full |
| units of BCG | BCG strain | p53 vector | shot of | # mice (BALB/c | time to form | progression of |
| injected^{a} | employed | used^{b} | BCG^{c} | mice) | tumor(days)^{e} | tumor(days)^{f} |

Experiment #1a:

| | | | | | |
|---|---|---|---|---|---|
| 3 × 10⁶, S.C. | Connaught | wt p53 | none | 0/5 | >122 |
| | | exons 5-11 | | | |

Experiment #1b:

| | | | | | | |
|---|---|---|---|---|---|---|
| 3 × 10⁶, S.C. | Connaught | mul p53 | none | 1/5 | 55 | 32 |
| | | exons 5-11 | | | | |

Experiment #1c:

| | | | | | | |
|---|---|---|---|---|---|---|
| 3 × 10⁶, S.C. | Connaught | mul p53 | 1 × 10⁶ | 1/5 | 49 | >73 |
| | | exons 5-11 | | | | |

Control Experiment #2a:

| | | | | | | |
|---|---|---|---|---|---|---|
| 3 × 10⁶, S.C. | Connaught | no p53 | none | 9/10 | 28 ± 19 | 20 |
| | | vector | | | | |
| | | only | | | | |

Control Experiment #2b:

| | | | | | | |
|---|---|---|---|---|---|---|
| none | none | none | none | 4/5 | 66 ± 7 | 21 |

Control Experiment #2c:

| | | | | | | |
|---|---|---|---|---|---|---|
| none | none | none | none | 8/10 | 28 ± 7 | 20 |

Experiment #3a:

| | | | | | | |
|---|---|---|---|---|---|---|
| 3 × 10⁶, S.C. | Pasteur | wt p53 | 3 × 10⁶, | 4/5 | 28 | >90 |
| | | full length, low | S.C. | | | |
| | | expression | | | | |

Experiment #3b:

| | | | | | | |
|---|---|---|---|---|---|---|
| 3 × 10⁶, S.C. | Pasteur | wt p53 | none | 5/5 | 31 ± 4 | >90 |
| | | full length, low | | | | |
| | | expression | | | | |

Experiment #3c:

| | | | | | | |
|---|---|---|---|---|---|---|
| 3 × 10⁶, S.C. | Pasteur | wt p53 | none | 10/10 | 8 ± 2 | 34 |
| | | full length, low | | | | |
| | | expression | | | | |

Notes to Table 4:
^{a}The primary immunization was given as a subcutaneous injection (SC) of 3 × 10⁶ BCG bacteria (colony forming units of bacteria) per mouse.
^{b}The wild-type p53 expression vector that expressed full length p53 expressed low levels of proteins (Pasteur strain of BCG) while the wild-type or mutant p53 vector that expressed only exons 5-11 of p53 protein expressed high (about 10-fold) levels of p53 protein.
^{c}When a booster injection was given, it was administered 17 days after the first injection as 1 × 10⁶ BCG cells, subcutaneous.
^{d}The challenge was carried out with tumorigenic 10(3) cells, which are isogenic with BALB/c mice, containing the 273 his to arg p53 mutant protein. The challenge was given 47 days after the primary injection.
^{e}Time to tumor formation is when the average of all tumors first appeared.
^{f}Time for full progression of the tumor is the average of the times all the tumors reached a size when the mouse had to be sacrificed. When the time indicated is greater than (>), the experiment is still in progress.

As shown in Table 4, the Connaught strain is better than the Pasteur strain at protecting mice from the tumor challenge, possibly due to a strain specific growth or presentation of the antigen or the higher levels of p53 antigens in the Connaught strain.

The results of Table 4 demonstrate that p53 expressed in BCG can protect from a tumor challenge with cells that have mutant p53, (the challenge was carried out with tumorigenic cells containing mutant p53 protein). Furthermore, in some cases (see experiments #1c, #3a, and #3b of Table 4) the Connaught and Pasteur strains with p53 protein slowed the progression of the tumor in mice.

In conclusion, the results of Table 4 demonstrate that immunizing with p53 protein presented in BCG does have an inhibiting effect upon the formation and growth of tumorigenic cells in BALB/c mice, and that mutant or wild-type p53 can be used as the antigen in BCG to protect from a tumor containing mutant p53.

### Example 3

Immunization with Recombinant Vaccines Expressing p53 in BCG

Mice were immunized with recombinant vaccines that expressed p53 protein. These vaccines were based on Mycobacterium tuberculosis typus bovis var. Calmette&Cuerin (BCG).

### Example 3A

### Materials and Methods

The (10)3-273.1NT24 cell line was derived from a nude mouse tumor induced by (10)3-273.1 cells (Dittmer, et al. (1993) Nature Genetics 4, 42-46). The cells express high levels of the human 273 allele of p53 and of the MHC-I allele H2-K^{d}. The cells were maintained in DMEM/10% FCS supplemented with 500-g/ml G418 and incubated at 37° C. and 5% CO₂.

Recombinant plasmids were generated using standard molecular biology techniques (Perbal. (1991) Methods in Molecular Biology (New York: Wiley). The pCMV-SN₃ vector expressing the human p53 cDNA is described in (Hinds, et al. (1990) Cell Growth Diff. 1, 571-580). The pMV261 and pMV262 expression plasmids are described in Stover, et al. (1993) J. Exp. Med. 178, 197-209.

Immunoblot analysis was carried out with extracts from BCG bacteria. The bacteria were lysed for 10 minutes on ice in lysis-buffer (10 mM Tris pH 7.4, 250 mM sucrose, 160 mM KCl, 50 mM ε-amino-caproic acid, 0.5% NP-40 supplemented to 3 mM β-mercaptoethanol, 1 mM PMSF and 0.28 TIU/ml aprotinin immediately prior to use) and sonicated. The insoluble particles were precipitated by spinning 20 min at maximum speed in an Eppendorf centrifuge. The protein concentration in the supernatant was determined by Biorad-assay (Biorad) and analyzed by 10% SDS-PAGE (Laemmli, (1970) Cancer Res. 53, 3468-3471). The proteins were then transferred to nitrocellulose (Amersham) by electroblotting for four hours in transfer buffer (25 mM Tris, 150 mM glycine) at ≦1.5 A. The membrane was stained with Ponceau S and the gel with Coomassie blue to check the transfer efficiency. The membrane was blocked for one hour in PBS/0.5% Tween 20/1% BSA. The primary antibody was diluted in PBS/0.5% Tween-20 and incubated at 4° C. for two hours. The p53-specific monoclonal antibody pab421, which is specific for a C-terminal epitope of p53 (Harlow, et al. (1981) J. Virology 39, 861-869), or mabl801 which is specific for human p53 (Banks, et al. (1986) J. Biochem. 159, 529-534), were used. After three washes 10 min in PBS/0.5% Tween-20 the membrane was incubated with peroxidase conjugated ant-mouse IgG (1:5000; Cappel) for 30 min at 4° C. The membrane was washed three times with PBS/0.5% Tween-20/0.1% Triton X-100 and the antibody complex was visualized using the ECL system (Amersham).

For immunoprecipitation, 1 mg soluble protein extract in 500 μl CLB-buffer was incubated with 25 μl protein A-sepharose (50% w/w protein A-sepharose, Pharmacia-LKB in 50 mM Tris pH 7.4, 5 mM EDTA, 0.5% NP-40, 150 mM NaCl) for 2 h at 4° C. and washed three times in SNNTE (50 mM Tris pH 7.4, 5 mM EDTA, 5% sucrose, 1% NP-40, 0.5 M NaCl). The beads were then analyzed by 10% SDS-PAGE and immunoblotting as before.

For immunization, Balb c/J mice were injected s.c. or i.v with the respective dose of recombinant BCG bacteria and challenged 6-8 weeks later with 10⁵ (10)3-273.1NT24 cells. Balb c/J mice were purchased from Jackson Laboratory, Maine, and housed under P3 conditions. The results are presented as the number of mice with tumors over the total number of mice injected. Tumors grew progressively until they were 0.5-1.0 cm in size and became necrotic. The animals were sacrificed by cervical dislocation at that time. Animals displaying no tumor after 3 standard deviations (SD) of the control group were considered tumor-free (Heitjan, (1993) Cancer Res. 53, 6042-6050). Tumor diameter was measured at weekly or biweekly intervals. All graphs of tumor growth plot the tumor diameter (d) in mm over time in days. This was fitted to an exponential function d(t)=tₒexp(K*t) least square approximation using Cricketgraph™. tₒ represents the time at which a tumor was initially visible, i.e. d(tₒ)≦1 mm. t_{1/2}=ln 2/K is called the doubling time. It is valid to display the diameter d=2*r rather than tumor volume V if we assume that for solid tumors, active tumor growth is limited to the outermost cells and thus r is exponentially dependent on time t (Edelstein-Keshet, (1987) Mathematical Models in Biology (New York: McGraw Hill).

### Example 3B

### Expression of Recombinant p53 ln BCG

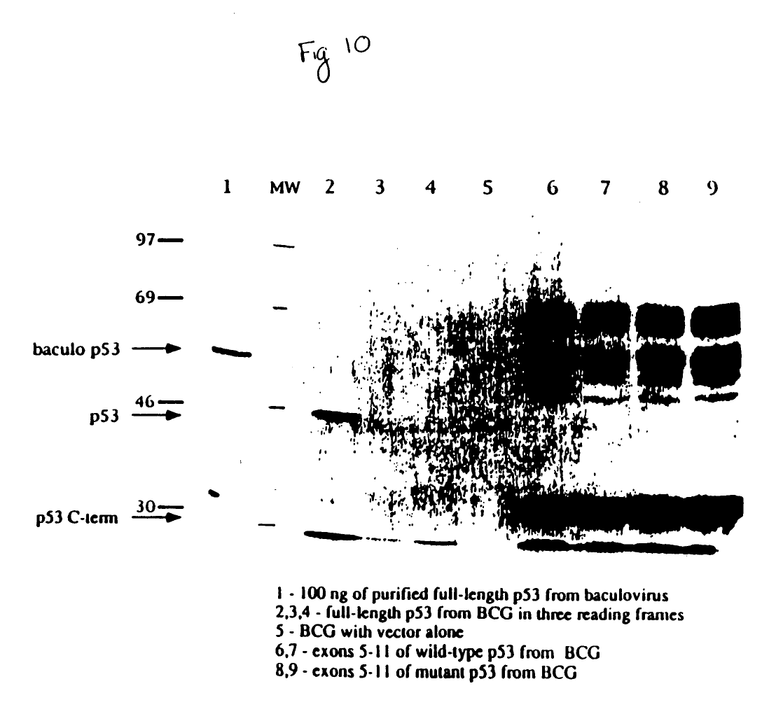

The human cDNA for wild-type p53 or the human 175 mutant allele was cloned in the BCG expression vector pMV261. The cloning was confirmed by restriction digest. Wild-type and mutant cDNA PvuII-EcoRI fragments from p53 vector (Hinds, et al., 1990) containing exons 5-11 (truncated) were fused to an eight amino acid leader of hsp60. The plasmids were amplified in E. coli and transformed into the Connaught strain of BCG. Expression was confirmed by immunoblot-analysis using the human p53-specific antibody pab1801 (FIG. 10).

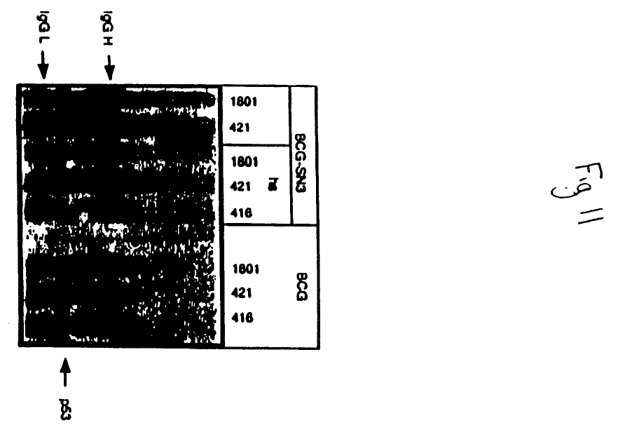

The BamHI fragment of SN₃ (Hinds, et al. (1990) Cell Growth Diff. 1, 571-580) containing the full-length p53 ORF was cloned into three different frames behind the BCG hsp60 promoter. This introduced a leader of 143 nucleotides between the promoter and the N-terminus of p53, which was subsequently found to contain a short ORF. This construct was also used to transform the Pasteur strain of BCG. The clone (BCG-1SN₃) expressed full-length p53 protein (FIG. 10). The expression level of full-length p53 protein was low compared to the expression level of the C-terminal fragments. C-terminal or N-terminal GST-fragments were expressed at higher level than full-length GST-p53 protein. Immunoprecipitation of bacterial extract using either pabl801 directed against an N-terminal epitope of p53 or pab421 directed against a C-terminal epitope of p53 confirmed that full-length p53 was produced by BCG-ISN₃ bacteria. The BCG hsp60 promoter was constitutively active and produced high levels of recombinant p53 protein even prior to heat shock (FIG. 11).

### Example 3C

### Immunization with Recombinant BCG by s.c. Injection

After the p53 expression in the recombinant BCG clones was confirmed, they were used in tumor protection assays. 3*10⁶ IU live recombinant BCG bacteria per animal were injected s.c. into Balb c/J mice (Table 5). Where indicated, groups of mice were boosted with the same dose. The boost did not affect the immunization efficacy. 7-8 weeks after the immunization, the animals were challenged with 1*10⁵ (10)3-273.1NT24 cells. Two sets of experiments were performed with different batches of BCG bacteria and with different batches of challenging cells.

The results were analyzed for three criteria: First, the tumor incidence was analyzed over the entire observation period of 248 or 229 days respectively. Animals immunized with the full-length p53 expressing BCG developed tumors (19/20), but in the second set of injections these tumors did not grow larger than 3 mm in diameter. The results with BCG expressing high levels of the truncated (i.e. exons 5-11) p53 were more promising. Comparing the tumor incidence of animals immunized with one of truncated p53 BCG constructs (2/15) to the tumor incidence of untreated mice or mice immunized with BCG vector alone (21/25) shows significant differences at p≦0.0000⁶ using the Yates χ² test for small sample numbers and p≦0.00004 using the Fisher two-tailed exact test. On the other hand no difference in tumor incidence between untreated animals (8/10) or animals immunized with BCG vector alone (9/10) could be detected.

TABLE 5
Table 5 shows the results of two sets of experiments
where BCG was injected s.c. Table 5 has three
categories: a) no immunization, b) immunization
with BCG Connaught and c) immunization with
BCG/Pasteur strain. Rows labeled (a) are from
the second experiment. The column entitled
“antigen” shows which p53 construct
was used for immunization. Tumor incidence
shows the number of animals which developed
tumors over a 248 day or a 229 day (experiment a)
period. The column entitled “time” shows the
mean time in days ± SD until the animals
developed 1 mm tumors. Animals which developed tumors
are included in this calculation. The column entitled
“progression” shows the time the tumor grew
exponentially from 1 mm until it became necrotic or
was sacrificed (usually d = 15-20 mm).
BCG Immunization:
All challenges with 1 * 10⁵(10)3-273.NT s.c cells
per mouse 7-8 weeks post immunization.

| | | | |
|---|---|---|---|
| antigen | tumor incidence | time | progression |

a) no immunization:

| | | | |
|---|---|---|---|
| — | 4/5 | 66 ± 7 | 21 ± 3 |
| —^{(a)} | 8/10 | 38 ± 43 | 20 ± 0 |

b) immunization with BCG Connaught strain

| | | | |
|---|---|---|---|
| mt, exon 5-11 | 2/5 | 55, 193 | 21 |
| mt, exon 5-11, boost | 0/5 | >248 | — |
| wt, exon 5-11 | 0/5 | | — |
| vector alone^{(a)} | 9/10 | 26 ± 9 | 31 ± 12 |

c) immunization with BCG Pasteur strain

| | | | |
|---|---|---|---|
| wt, exon 1-11, boost | 4/5 | 30 ± 3 | 218 ± 3 |
| wt, exon 1-11 | 5/5 | | |
| wt, exon 1-11^{(a)} | 10/10 | 7 ± 1 | 34 ± 1 |

^{(a)}different date of challenge

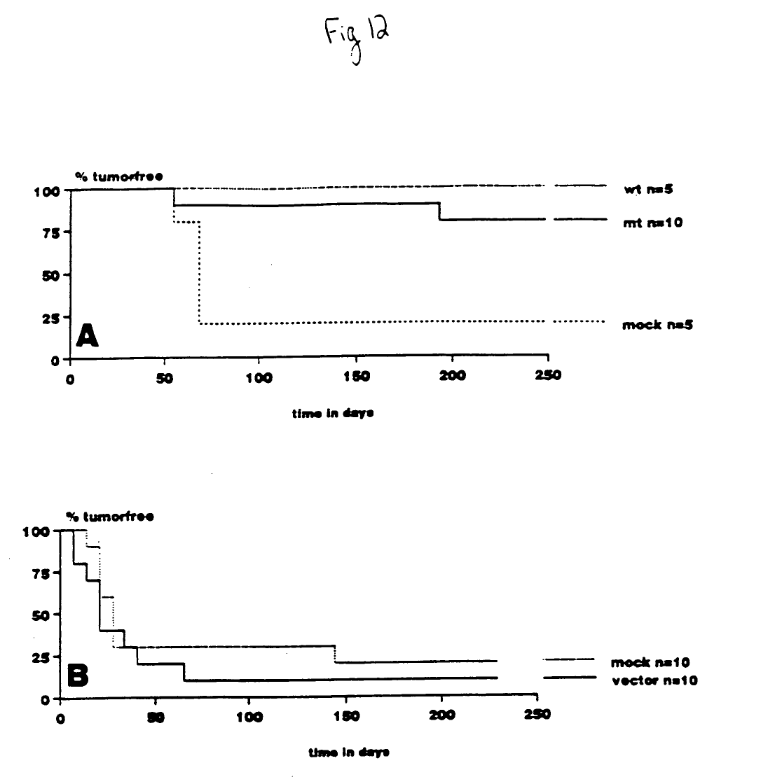

Secondly, the time of tumor onset was compared between animals immunized with either one of truncated p53 constructs (n=15) or not treated (n=5). The difference in tumor onset between treated and untreated animals was significant (p<0.0007 using an independent t-test with pooled variance). The significance level will go up further, since the animals which were protected are still alive. Immunization with BCG alone did not delay tumor onset (p>0.4 using an independent t-test with either separate or pooled variance). This result is presented in a Kaplan-Meyer plot of percent tumor-free animals over time (FIG. 12). The immunizations which involved full-length p53-expressing BCG were less conclusive. In set a) animals immunized with full-length BCG developed tumors earlier than those immunized with the vector alone. Although this observation was significant to p<0.006 (independent t-test with separate variance) it could not be repeated in the second set of immunizations (independent t-test with separate variance p=0.27).

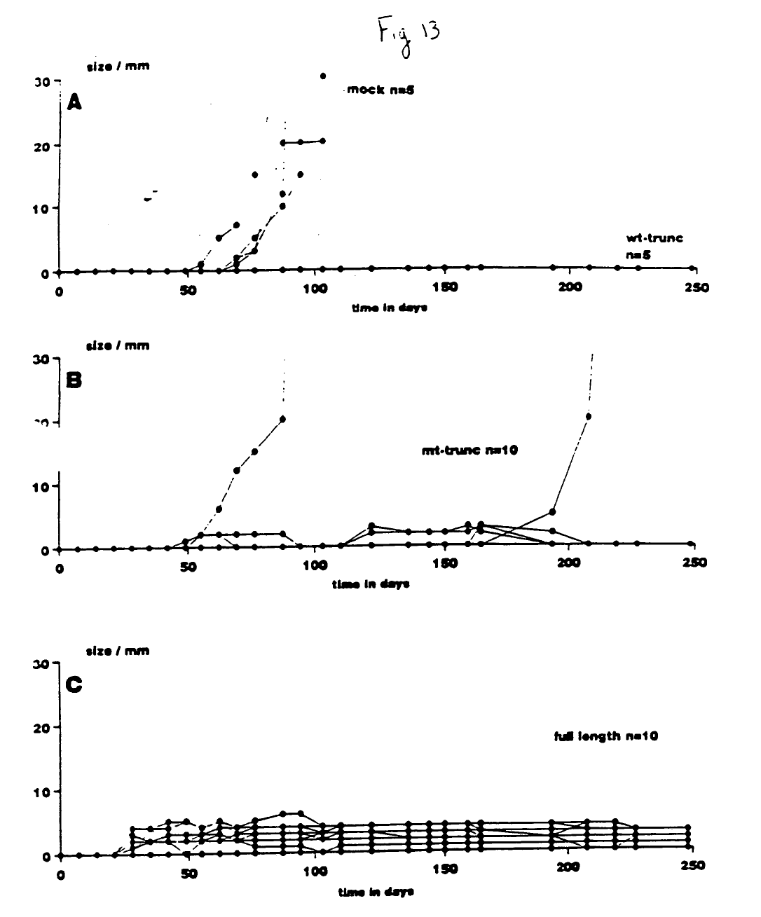

Thirdly, the time of tumor progression was analyzed. It was similar for untreated animals and those immunized with the BCG vector control (p=0.86 in a paired t-test using data from set (a)). Two animals developed tumors after immunization with BCG expressing truncated p53. Here the tumors grew exponentially with a progression time of 21 days. This progression time is well within the progression time (21±3 days) seen in the untreated group. Animals immunized with BCG expressing full-length p53 protein showed different responses in the two sets of experiments. In the first set of experiments the animals developed tumors at the same incidence (10/10) as untreated animals but at an earlier time (7±1 days compared to 26±9) days, with p<0.006 in an independent t-test with separate variances). Those tumors progressed at the same rate as the tumors in animals immunized with the BCG vector control (p>0.46 in an independent t-test with separate variances). In the second set of experiments, the animals showed the same incidence and initial time of tumor onset but the lesions never progressed (p<0.00001 in an independent t-test with separate variances, FIG. 13). It is unclear whether these lesions represent tumors which stopped growing or scar tissue as a result of BCG-induced local inflammation.

### Example 4

### Immunization with Recombinant ALVAC

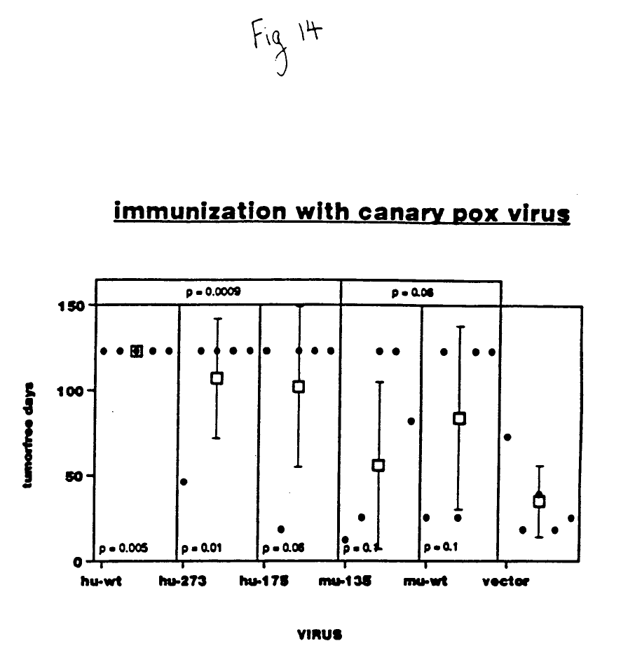

5 mice each were injected s.c. with 5*10⁷ avipoxvirus genus canary poxvirus (ALVAC) particles, boosted 28 days after immunization and injected s.c. with 10⁵ (10)3-273.1NT24 cells 52 days after immunization. Tumorigenicity was analyzed as before (FIG. 14). Mice immunized with the vector alone developed tumors at 35t21 days. Mice immunized with ALVAC expressing murine wild-type p53 or the murine 135 allele of p53 developed tumors later (70±50 and 55±50 days, respectively). Mice immunized with ALVAC expressing human wild-type p53, the human mutant p53 allele 175 or the human mutant p53 allele 273 were protected against (10)3-273.1NT24 induced tumors (p≦0.001 for n=15 mice using the Mann-Whitney U Test). Only 1 mouse out of 5 immunized with ALVAC expressing either the 175 or 273 allele of p53 developed a tumor. None of the mice immunized with ALVAC expressing the wild-type human p53 allele developed a tumor.

1. A vaccine composition comprising a mutant p53 protein in a form that, when presented to the immune system of a mammal, induces an effective immune response.

2. A vaccine composition according to claim 1 wherein the composition also comprises a pharmaceutically acceptable medium.

3. A vaccine composition according to claim 1 wherein the form is either the mutant p53 protein on the surface of an antigen presenting cell or the mutant p53 protein combined with a pharmaceutically acceptable adjuvant.

4. A vaccine composition according to claim 3 wherein the form is the mutant p53 protein on the surface of an antigen presenting cell.

5. A vaccine composition according to claim 3 wherein the form is the mutant p53 protein combined with a pharmaceutically acceptable adjuvant.

6. A vaccine composition according to claim 4 wherein the antigen presenting cell is a eucaryotic cell.

7. A vaccine composition according to claim 6 wherein the eucaryotic cell is a dendritic cell, a major histocompabbility complex Class II positive macrophage or a monocyte.

8. A vaccine composition according to claim 7 wherein the antigen presenting cell is a dendritic cell.

9. A vaccine composition according to claim 8 wherein the dendritic cell is a recombinant dendritic cell that expresses exogenous DNA encoding mutant p53 protein on its surface.

10. A vaccine composition according to claim 5 wherein the pharmaceutically acceptable adjuvant is a bacterial cell.

11. A vaccine composition according to claim 10 wherein the bacterial cell is bacille Calmette-Guerin.

12. A vaccine composition according to claim 11 wherein the bacille Calmette-Guerin is a recombinant bacille Calmette-Guerin that expresses exogenous DNA encoding mutant p53 protein.

13. A method of inhibiting the growth of tumors in mammals comprising treating a mammal with an immunologically effective amount of a vaccine composition comprising a mutant p53 protein in a form that, when presented to the immune system of a mammal, induces an effective immune response.

14. The method of claim 13 wherein the vaccine composition also comprises a pharmaceutically acceptable medium.

15. The method of claim 13 wherein the form is either the mutant p53 protein on the surface of an antigen presenting cell or the mutant p53 protein combined with a pharmaceutically acceptable adjuvant.

16. The method according to claim 15 wherein the form is the mutant p53 protein on the surface of an antigen presenting cell.

17. The method according to claim 15 wherein the form is the mutant p53 protein combined with a pharmaceutically acceptable adjuvant.

18. The method of claim 16 wherein the antigen presenting cell is a eucaryotc cell.

19. The method of claim 18 wherein the eucaryotc cell is a dendritic cell, a major histocompabbility complex Class II positive macrophage or a monocyte.

20. The method of claim 19 wherein the antigen presenting cell is a dendritic cell.

21. The method of claim 20 wherein the dendritic cell is a recombinant dendritic cell that expresses exogenous DNA encoding mutant p53 protein.

22. The method of claim 17 wherein the pharmaceutically acceptable adjuvant is a bacterial cell.

23. The method of claim 22 wherein the bacterial cell is bacille Calmette-Guerin.

24. The method of claim 23 wherein the bacille Calmette-Guerin is a recombinant bacille Calmette-Guerin that expresses exogenous DNA encoding mutant p53 protein.

25. A recombinant antigen presenting cell that expresses exogenous DNA encoding mutant p53 protein.

26. A vaccine composition comprising a wild-type p53 protein in a form that, when presented to the immune system of a mammal, induces an effective immune response.

27. A vaccine composition according to claim 26 wherein the composition also comprises a pharmaceutically acceptable medium.

28. A vaccine composition according to claim 26 wherein the form is either the wild-type p53 protein on the surface of an antigen presenting cell or the wild-type p53 protein combined with a pharmaceutically acceptable adjuvant.

29. A vaccine composition according to claim 28 wherein the form is the wild-type p53 protein on the surface of an antigen presenting cell.

30. A vaccine composition according to claim 28 wherein the form is the wild-type p53 protein combined with a pharmaceutically acceptable adjuvant.

31. A vaccine composition according to claim 29 wherein the antigen presenting cell is a eucaryotc cell.

32. A vaccine composition according to claim 31 wherein the eucaryotic cell is a dendritic cell, a major histocompatibility complex Class II positive macrophage or a monocyte.

33. A vaccine composition according to claim 32 wherein the antigen presenting cell is a dendritic cell.

34. A vaccine composition according to claim 33 wherein the dendritic cell is a recombinant dendritic cell that expresses exogenous DNA encoding wild-type p53 protein on its surface.

35. A vaccine composition according to claim 30 wherein the pharmaceutically acceptable adjuvant is a bacterial cell.

36. A vaccine composition according to claim 35 wherein the bacterial cell is bacille Calmette-Guerin.

37. A vaccine composition according to claim 36 wherein the bacille Calmette-Guerin is a recombinant bacille Calmette-Guerin that expresses exogenous DNA encoding wild-type p53 protein.

38. A method of inhibiting the growth of tumors in mammals comprising treating a mammal with an immunologically effective amount of a vaccine composition comprising a wild-type p53 protein in a form that when presented to the immune system of a mammal, induces an effective immune response.

39. The method of claim 38 wherein the vaccine composition also comprises a pharmaceutically acceptable medium.

40. The method of claim 38 wherein the form is either the wild-type p53 protein on the surface of an antigen presenting cell or the wild-type p53 protein combined with a pharmaceutically acceptable adjuvant.

41. The method according to claim 40 wherein the form is the wild-type p53 protein on the surface of an antigen presenting cell.

42. The method according to claim 40 wherein the form is the wild-type p53 protein combined with a pharmaceutically acceptable adjuvant.

43. The method of claim 41 wherein the antigen presenting cell is a eucaryotc cell.

44. The method of claim 43 wherein the eucaryotc cell is a dendritic cell, a major histocompatibility complex Class II positive macrophage or a monocyte.

45. The method of claim 44 wherein the antigen presenting cell is a dendritic cell.

46. The method of claim 45 wherein the dendritic cell is a recombinant dendritic cell that expresses exogenous DNA encoding wild-type p53 protein.

47. The method of claim 42 wherein the pharmaceutically acceptable adjuvant is a bacterial cell.

48. The method of claim 47 wherein the bacterial cell is bacille Calmette-Guerin.

49. The method of claim 48 wherein the bacille Calmette-Guerin is a recombinant bacille Calmette-Guerin that expresses exogenous DNA encoding wild-type p53 protein.

50. A recombinant antigen presenting cell that expresses exogenous DNA encoding wild-type p53 protein.

51. A vaccine composition according to claim 1 wherein the mutant p53 protein is a fragment expressed by a truncated mutant p53 gene.

52. A vaccine composition according to claim 51 wherein the truncated mutant p53 gene lacks exons 1-4.

53. A vaccine composition according to claim 51 wherein the truncated mutant p53 gene comprises exons 5-11.

54. A method according to claim 13 wherein the mutant p53 protein is a fragment expressed by a truncated mutant p53 gene.

55. A method according to claim 54 wherein the truncated mutant p53 gene lacks exons 1-4.

56. A method according to claim 54 wherein the truncated mutant p53 gene comprises exons 5-11.

57. A recombinant antigen presenting cell according to claim 25 wherein the mutant p53 protein is a fragment expressed by a truncated mutant p53 gene.

58. A recombinant antigen presenting cell according to claim 57 wherein the truncated mutant p53 gene lacks exons 1-4.

59. A recombinant antigen presenting cell according to claim 57 wherein the truncated mutant p53 gene comprises exons 5-11.

60. A vaccine composition according to claim 26 wherein the wild-type p53 protein is a fragment expressed by a truncated wild-type p53 gene.

61. A vaccine composition according to claim 60 wherein the truncated wild-type p53 gene lacks exons 1-4.

62. A vaccine composition according to claim 60 wherein the truncated wild-type p53 gene comprises exons 5-11.

63. A method according to claim 38 wherein the wild-type p53 protein is a fragment expressed by a truncated wild-type p53 gene.

64. A method according to claim 63 wherein the truncated wild-type p53 gene lacks exons 1-4.

65. A method according to claim 63 wherein the truncated wild-type p53 gene comprises exons 5-11.

66. A recombinant antigen presenting cell according to claim 50 wherein the wild-type p53 protein is a fragment expressed by a truncated wild-type p53 gene.

67. A recombinant antigen presenting cell according to claim 66 wherein the truncated wild-type p53 gene lacks exons 1-4.

68. A recombinant antigen presenting cell according to claim 66 wherein the truncated wild-type p53 gene comprises exons 5-11.
